(19) United States
(12) Patent Application Publication
(10) Pub. No.: US 2020/0386714 A1
(43) Pub. Date: Dec. 10, 2020

(54) Electromagnetic Probe To Detect Discontinuities In Metal Joints

(71) Applicant: GM GLOBAL TECHNOLOGY OPERATIONS LLC, Detroit, MI (US)

(72) Inventors: Amberlee S. HASELHUHN, Troy, MI (US); Megan E. MCGOVERN, Royal Oak, MI (US); Teresa J. RINKER, Royal Oak, MI (US); Blair E. CARLSON, Ann Arbor, MI (US)

(21) Appl. No.: 16/430,656

(22) Filed: Jun. 4, 2019

(51) Int. Cl. G01N 27/90; G01N 33/207

(52) U.S. Cl. CPC G01N 27/9093; G01N 33/207; G01N 27/902

(57) ABSTRACT

A probe for detecting discontinuities includes: a body portion; a head portion; one or more inductor coils located in the head portion and configured to: receive power from a power source; when power is received, induce an eddy current in a joint where a first metal component is joined with a second metal component; based on the induced eddy current, output a signal indicative of an inductance of the one or more inductor coils; and a plurality of positioning devices configured to maintain the one or more inductor coils approximately a predetermined distance from the surface.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to detecting cracks in welded metal joints and more particularly to electromagnetic probes for detecting cracks in welded metal joints.

Welding is a process that joins two or more pieces of material together, such as two pieces of metal. Welding involves use of heat to melt two pieces together and allowing the pieces to cool to cause the pieces to fuse together. This is in contrast with other types of joining techniques, such as brazing and soldering, which do not melt the base pieces to be joined.

Different types of heat sources can be used for different types of welding. For example, electricity is used in arc welding. One type of arc welding is resistance welding. One or more lasers are used to perform laser welding. Other types of welding include, but are not limited to, electron beam welding, friction welding, and ultrasound welding.

Resistance welding involves the generation of heat by passing current through the resistance caused by the contact between two or more metal surfaces. Small pools of molten metal are formed at the weld area as high current (e.g., 1000-100,000 Amps) is passed through the metal.

Spot welding (or resistance spot welding (RSW)) is a resistance welding method used to join overlapping metal components (e.g., sheets). Two electrodes simultaneously pass current through the sheets to weld the components together. The locations where current is passed through the components and the components are joined together can be referred to as spot welds.

### SUMMARY

In a feature, a probe for detecting discontinuities includes: a body portion; a head portion; one or more inductor coils located in the head portion and configured to: receive power from a power source; when power is received, induce an eddy current in a joint where a first metal component is joined with a second metal component; based on the induced eddy current, output a signal indicative of an inductance of the one or more inductor coils; and a plurality of positioning devices configured to maintain the one or more inductor coils approximately a predetermined distance from the surface.

In further features, the positioning devices include a plurality of extensions that extend only radially outwardly from the head portion.

In further features, the one or more inductor coils are located in one of the extensions.

In further features, the plurality of extensions include at least three extensions that extend only radially outwardly from the head portion, where the at least three separate extensions are located equidistantly around the head portion.

In further features, the plurality of extensions define a circle having a first diameter that is greater than a second diameter of the joint.

In further features, the joint includes spot weld formed by resistance spot welding (RSW).

In further features, a central portion is rotatable within the body portion, and the head portion is coupled to and rotates with the central portion.

In further features, the plurality of positioning devices include rollers.

In further features, the rollers include one of ball casters, ball transfers, and Hudson bearings.

In further features, the rollers are spring loaded such that the rollers are biased away from the head portion.

In further features, the head portion defines a circle having a diameter, all of the rollers are located within the diameter of the circle, and the diameter of the circle is one of less than and greater than a second diameter of the joint.

In further features, the head portion defines a circle having a diameter, and a plurality of the rollers are located outside of the diameter of the circle.

In further features, a rotating portion is configured to rotate radially inwardly and radially outwardly, and the one or more inductor coils are located in the rotating portion.

In further features, an actuator is configured to rotate the rotating portion back and forth from a first predetermined radially inward position and a second predetermined radially outward position.

In further features, the actuator includes one of: a motor; and a rotating member and a multiple bar linkage that is coupled to the rotating member.

In further features, the probe further includes a protective wear plate.

In further features, the probe includes a plurality of legs that extend radially outwardly from the probe and that are configured to extend toward the joint.

In further features, the legs define a circle having a first diameter that is one of less than and greater than a second diameter of the joint.

In a feature, a method of detecting discontinuities includes: locating a probe within a joint on a first metal component where the first metal component is joined with a second metal component, where the probe includes: one or more inductor coils configured to: receive power from a power source; when power is received, induce an eddy current in the joint; and based on the induced eddy current, output a signal indicative of an inductance of the one or more inductor coils; and a plurality of positioning devices configured to maintain the one or more inductor coils approximately a predetermined distance from the joint; and rotating the probe within the joint while maintaining the positioning devices in contact with at least one of: the joint; and the first metal component.

In further features, the method further includes: generating a two-dimensional map of a region of interest within the joint based on the signal during the rotation of the probe within the joint.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 includes a cross-sectional view including an example spot weld formed by resistance spot welding (RSW);

FIG. 2 includes an example enhanced top view of a spot weld including two cracks;

FIG. 3 includes a functional block diagram of an example crack detection system including a probe;

FIG. 4 includes an example graph of impedance;

FIG. 5 includes a cross-sectional side view including an example implementation of the probe of FIG. 3 and a spot weld;

FIG. 6 includes a top view including the probe of FIG. 3 and the spot weld;

FIG. 7 includes a side view including an example implementation of the probe of FIG. 3 and the spot weld;

FIG. 8 includes a top view including the example implementation of the probe of FIG. 3 and the spot weld;

FIG. 9 includes a perspective side view including an example implementation of the probe of FIG. 3 and the spot weld;

FIG. 10 includes a top view including an example implementation of the probe of FIG. 3 and the spot weld;

FIG. 11 includes a perspective side view including an example implementation of the probe of FIG. 3 and the spot weld;

FIG. 12 includes a cross-sectional side view including an example implementation of the probe of FIG. 3 and the spot weld;

FIG. 13 includes a perspective side view including an example implementation of the probe of FIG. 3 and the spot weld;

FIG. 14 includes a perspective side view including an example implementation of the probe of FIG. 3 and the spot weld;

FIG. 15 includes a perspective side view including an example implementation of the probe of FIG. 3 and the spot weld;

FIGS. 16 and 17 include illustrations of example arrays of multiple coils; and

FIG. 18 includes an example two-dimensional (2-D) eddy current map showing a crack in the circumference of a spot weld.

In the drawings, reference numbers may be reused to identify similar and/or identical elements.

### DETAILED DESCRIPTION

Resistance spot welding (RSW) can be used to join overlapping metal components (e.g., sheets). To perform RSW, an electric current is passed between two welding electrodes and through a workpiece stack-up including two or more components. Resistance to the flow of electric current rapidly heats up the more thermally and electrically resistive workpiece. This creates a molten weld pool at the faying interface between two or more components which, upon termination of current flow, solidifies into a weld joint that bonds the two or more metal components together.

Cracks may occur at one or more spot welds. Destructive analysis can be used to observe cracks at spot welds. However, destructive analysis is time consuming and destroys parts. Non-destructive techniques include surface altering methods such as liquid dye pentrant, which is undesirable since the surface should not be altered prior to painting. Cracks at spot welds may interfere with later processing of the joined components, such as painting. The cracks may also decrease mechanical integrity of a joint. Cracks are stress concentrators that can cause joints to prematurely fail in the presence of an applied load. Further, cracks can accelerate the effects of corrosion and lead to corrosion-related failure of the joint.

The present application describes a non-destructive probe that can be used to detect discontinuities (e.g., cracks, surface cavities, etc.) at spot welds and other types of metal joints. The probe includes a plurality of positioning devices configured to maintain an approximately constant distance between inductor coils of the probe and a surface of a spot weld. By maintaining the approximately constant distance, the probe measures an approximately constant inductance in the absence of cracks while the inductor coils are moved around the spot weld. Crack detection may therefore be made more reliable as a change (e.g., an increase) in the inductance measured by the probe is more likely to be due to the presence of a crack than a change in the distance between the inductor coils and the surface of the spot weld. A change in the distance between the inductor coils and the surface of the spot weld may cause a proportional change in the inductance measured by the probe.

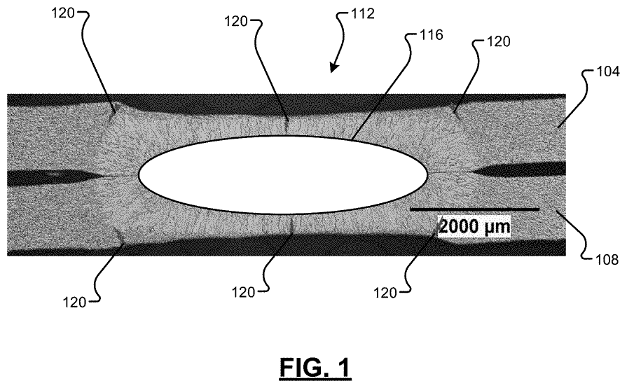

FIG. 1 includes a cross-sectional view including a first metal sheet 104 and a second metal sheet 108 joined at a weld 112 (e.g., a spot weld) formed by welding, such as resistance spot welding (RSW). Other forms of welding include, but are not limited to, laser welding, friction welding, etc. The RSW results in a weld nugget 116 being formed where the first and second metal sheets 104 and 108 are joined. The first and second metal sheets 104 and 108 may be, for example, generation 3 steel sheets that are zinc coated or another suitable type of metal sheets. While the example of joining the first and second metal sheets 104 and 108 is provided, the present application is also applicable to joining more than two metal components and joining of metal components other than metal sheets.

One or more cracks may form at the weld 112. For example, example cracks 120 are depicted schematically in FIG. 1. Weld spots, however, may include zero or more than zero cracks.

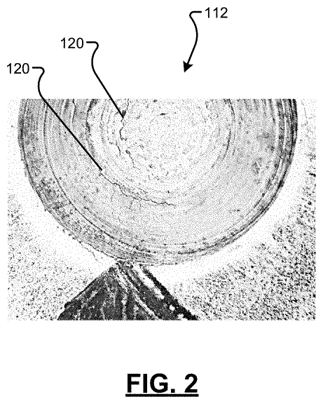

FIG. 2 includes an example enhanced top view of the weld 112 including two of the cracks 120. The cracks 120 may form, for example, due to liquid metal embrittlement (LME).

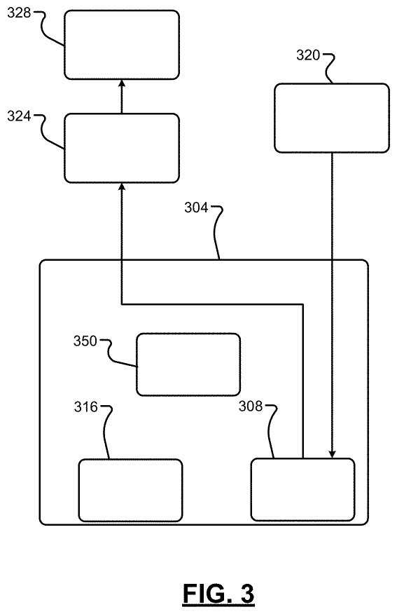

FIG. 3 includes a functional block diagram of an example crack detection system including a probe 304. The probe 304 is configured to locate one or more inductor coils 308 within the weld 112, such as within a heat affected zone (HAZ) region of the weld 112.

The probe 304 includes positioning devices 316 configured to maintain an approximately constant distance (or liftoff) between the inductor coils 308 and the surface of the weld 112 while the inductor coils 308 are moved (e.g., rotated) around the weld 112. Approximately may mean within a predetermined tolerance, such as +/−25 percent of an average. Examples of the positioning devices 316 are described further below.

The inductor coils 308 are electrically connected in parallel. A power supply module 320 applies power to the inductor coils 308. For example, the power supply module 320 may apply alternating current (AC) power having a constant magnitude and frequency to the inductor coils 308. The inductor coils 308 generate magnetic field based on the power applied to the inductor coils 308.

The magnetic field induces an eddy current in the metal of the weld 112. At least one of (a) the number of inductor coils 308 used, (b) the number of turns of each of the inductor coils 308, (c) the radius of the inductor coils 308, (d) the magnitude of the power, and (e) the frequency of the power can be set or selected based on at least one of the materials joined, the joining method used, the welding electrode profile, and the size (e.g., diameter) of the weld 112.

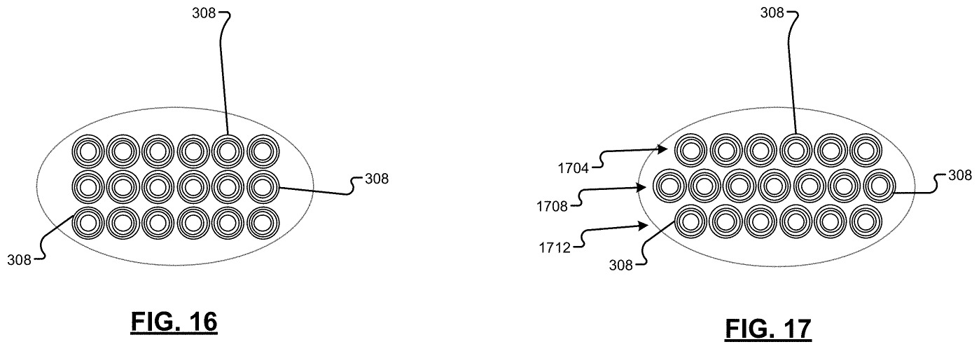

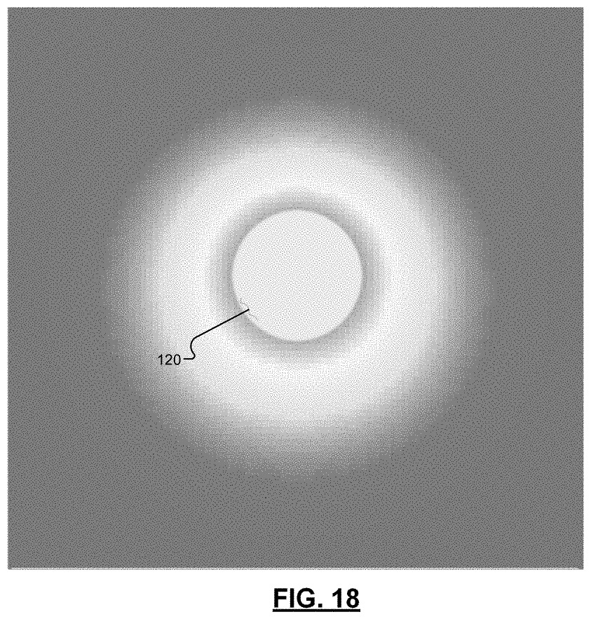

A two-dimensional (2-D) area scan can be captured by scanning with a single point probe (having one inductor coil) and measuring location with one or more x-y encoders. Alternatively, an array of probes (e.g., FIGS. 16-17 below) can be used to directly capture a 2-D area of a region of interest, where the 2-D area is defined by the area of the probe array. To capture an area that is larger than the area of the probe array, the probe can be moved and the location captured with the use of one or more x-y encoders. FIG. 18 includes an example 2-D area captured including a crack 120 in the circumference of a weld.

Changes in the eddy current in the metal of the weld 112 cause changes the magnetic field and, therefore, an inductance of the inductor coils 308. The inductance of the inductor coils 308 changes at locations where cracks or other discontinuities are present in the weld 112. The inductance of the inductor coils 308 also changes as the distance between the inductor coils 308 and the surface of the weld 112 changes. Because the positioning devices 316 maintain the approximately constant distance (or liftoff) between the inductor coils 308 and the surface of the weld 112 during movement of the inductor coils 308, changes in the impedance are more likely to be attributable to cracks or other discontinuities in the weld 112.

A measurement module 324 measures the impedance of the inductor coils 308. A crack detection module 328 detects cracks in the weld 112 based on the impedance of the inductor coils 308. For example, the crack detection module 328 may detect the presence of a crack in the spot weld when the impedance changes (increases or decreases) by more than a predetermined amount.

In various implementations, the (same) inductor coils 308 may both receive power from the power supply module 320 and be used to measure the impedance. Alternatively, the inductor coils 308 may include a first set of one or more inductor coils that are electrically connected in parallel and that receive power from the power supply module 320. The inductor coils 308 also include a second set of one or more inductor coils that are electrically connected in parallel and that are used to measure the impedance.

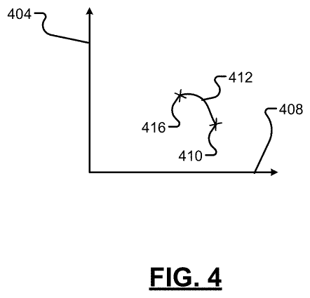

FIG. 4 includes an example graph of inductive reactance 404 versus resistance 408 as the inductor coils 308 and liftoff is maintained constant within the weld 112 during rotation within the weld 112. Because the positioning devices 316 maintain the approximately constant distance (liftoff) during the rotation, as an example, the impedance remains approximately constant when no cracks are present in the weld 112. Point 410 corresponds to an impedance 412 when no crack is present. Point 416 illustrates that the impedance 412 changes when a crack is present. Because the impedance 412 remains approximately constant when no cracks are present, the presence of cracks is more readily identifiable via the impedance 412.

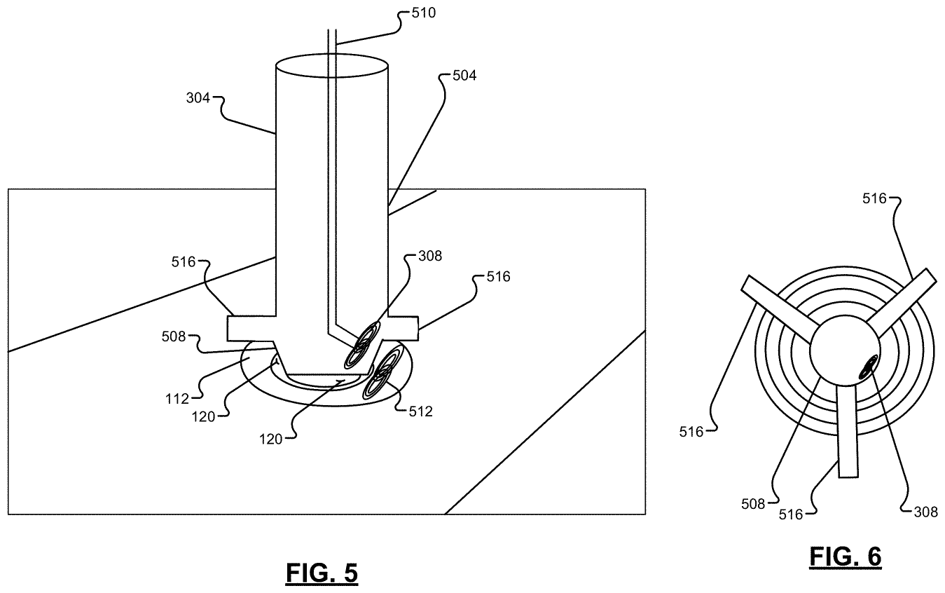

FIG. 5 includes a cross-sectional side view including an example implementation of the probe 304 of FIG. 3 and the weld 112. FIG. 6 includes a top view including the example implementation of the probe 304 of FIG. 3 and the weld 112. The probe 304 includes a body portion 504 and a head portion 508. While the body portion 504 is illustrated as being cylindrical, the body portion 504 may have another suitable shape.

The inductor coils 308 are located within the head portion 508, such as near a radially outer edge of the head portion 508. An example illustration of the eddy current induced by the inductor coils 308 is shown as 512 in FIG. 5. Wires 510 connecting the inductor coils 308 to the power supply module 320 and the measurement module 324 extend through the body portion 504. As shown in the example of FIG. 5, the body portion 504 may be fixed to the head portion 508. In this example, the inductor coils 308 may be rotated within the weld 112 by rotating the body portion 504.

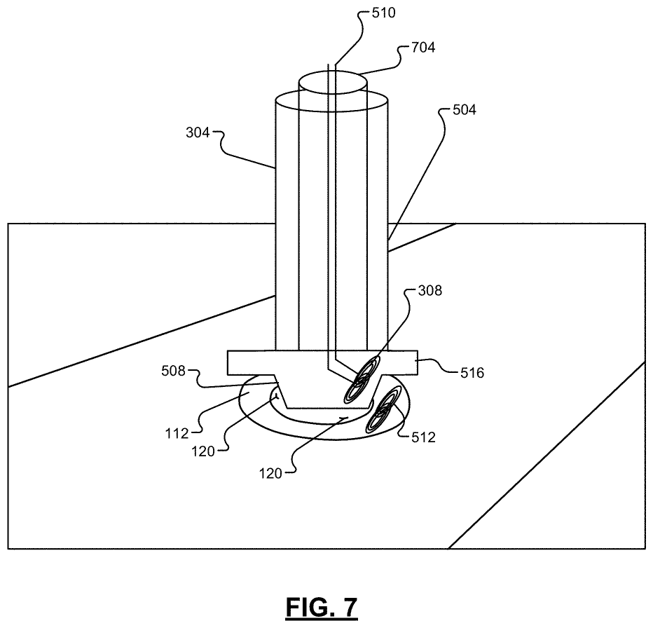

FIG. 7 includes a side view including an example implementation of the probe 304 of FIG. 3 and the weld 112. In the example of FIG. 7, a central portion 704 is located within the body portion 504 and is rotatable. The head portion 508 is coupled to and rotates with the central portion 704. In this example, the inductor coils 308 may be rotated within the weld 112 by rotating the central portion 704. The body portion 504 may remain stationary and not rotate.

As shown in the examples of FIGS. 5-7, the positioning devices 316 include extensions 516 that extend radially outwardly from the head portion 508. The extensions 516 extend radially outwardly such that a radial length of the extensions 516 is greater than a radial distance between a center of the weld 112 and an outer edge of the weld 112. In other words, a diameter of a circle defined by the extensions 516 is greater than a diameter of a circle defined by the weld 112. Thus, the extensions 516 rest outside of the weld 112 on the surface of one of the metal sheets.

While the example of three extensions is provided, the probe 304 may include two or more extensions. The extensions 516 may maintain the approximately constant distance between the inductor coils 308 and the surface of the weld 112 during rotation of the inductor coils 308. In various implementations, one, more than one, or all of the extensions 516 may be omitted.

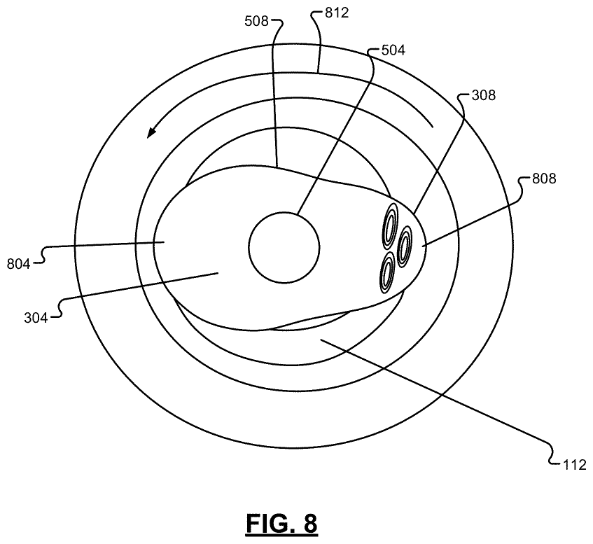

FIG. 8 includes a top view including the example implementation of the probe 304 of FIG. 3 and the weld 112. In the example of FIG. 8, the head portion 508 is oblong and includes at least two portions 804 and 808 that extend radially outwardly to outside of the weld 112 on the surface of one of the metal sheets. While the example of two portions is provided, the head portion 508 may include more than two portions that extend outside of the weld 112. By resting on the surfaces of the metal sheet outside of the weld 112, the portions 804 and 808 may maintain the approximately constant distance between the inductor coils 308 and the surface of the weld 112 during rotation of the inductor coils 308. Example rotation is illustrated by arrow 812.

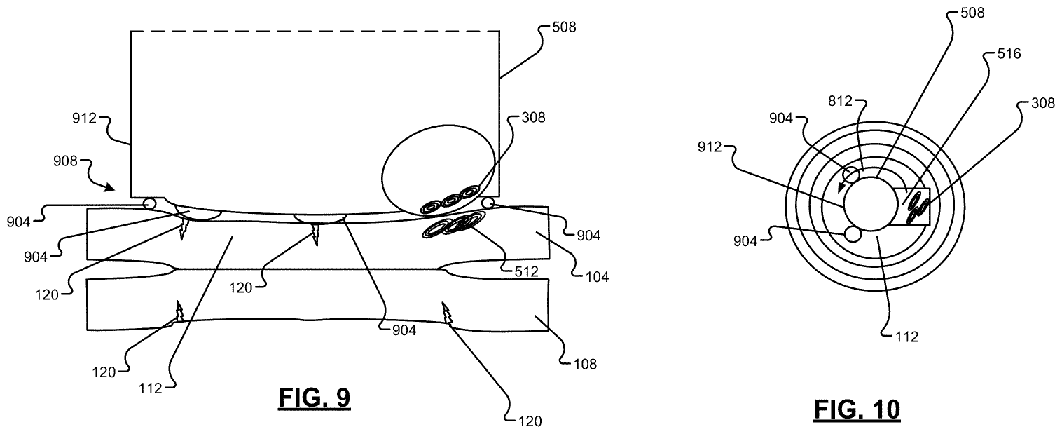

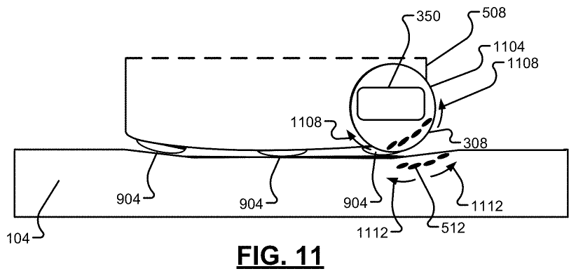

FIG. 9 includes a perspective side view including an example implementation of the probe 304 of FIG. 3 and the weld 112. FIG. 10 includes a top view including an example implementation of the probe 304 of FIG. 3 and the weld 112.

As shown the examples of FIGS. 9-10, the positioning devices 316 include rollers 904 located at the distal end 908 of the head portion 508. The rollers 904 may be spring-loaded and may be, for example, ball casters, ball transfers, Hudson bearings, or another suitable type of roller. The rollers 904 may maintain the approximately constant distance between the inductor coils 308 and the surface of the weld 112 during rotation of the inductor coils 308. The rollers 904 may be non-electrically conductive (e.g., polymer, nylon, etc.) or electrically conductive.

As shown in FIG. 9, one, more than one, or all of the rollers 904 may be located radially inwardly of a radially outer edge 912 of the head portion 508. As shown in FIG. 10, one, more than one, or all of the rollers 904 may be located radially outwardly of the radially outer edge of the head portion 508. While two of the rollers 904 are shown in the example of FIG. 10, a third roller may be located underneath the extension 516. In the example of FIG. 10, the extension 516 may not rest on the surface of one of the metal sheets. Instead, the rollers maintain the extension 516 separated from the surface of the one of the metal sheets. In various implementations, the rollers 904 may define a circle that has a diameter that is less than the diameter of the weld 112. Additionally or alternatively, the rollers 904 may define a circle that has a diameter that is greater than the diameter of the weld 112.

Referring back to FIG. 3, in various implementations, the probe 304 may include an actuator 350 that actuate the inductor coils 308 radially inwardly and radially outwardly. For example only, the actuator 350 may repeatedly actuate the inductor coils 308 back and forth between a predetermined radially inward position and a predetermined radially outward position.

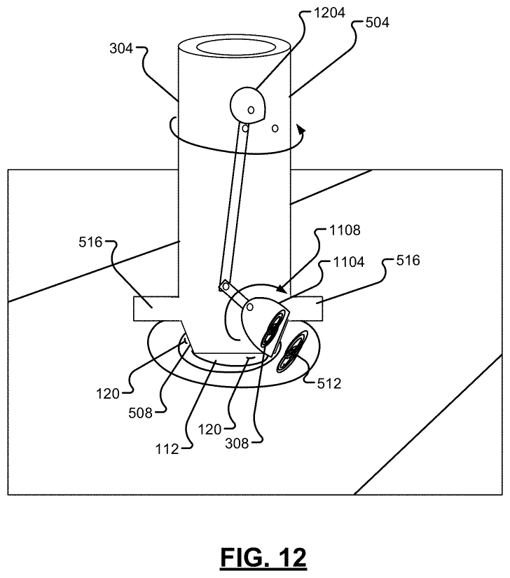

FIG. 11 includes a perspective side view including an example implementation of the probe 304 of FIG. 3 and the weld 112. The inductor coils 308 may be fixed within a rotating portion 1104. The actuator 350 may rotate the rotating portion 1104 back and forth radially inwardly and radially outwardly, as illustrated by arrows 1108.

The radially movement of the inductor coils 308 causes the induced eddy current 512 to move radially inwardly and outwardly within the weld 112 as illustrated by arrows 1112. Thus, a larger portion of the weld 112 can be scanned for cracks.

The actuator 350 may include, for example, an electric motor (e.g., a servomotor) configured to actuate the rotating portion 1104 back and forth between the predetermined radially outward position and the predetermined radially inward position. Alternatively, as shown in FIG. 12, the actuator 350 may include a rotating member (e.g., a camshaft) and a multiple bar linkage. FIG. 12 includes a cross-sectional side view including an example implementation of the probe 304 of FIG. 3 and the weld 112.

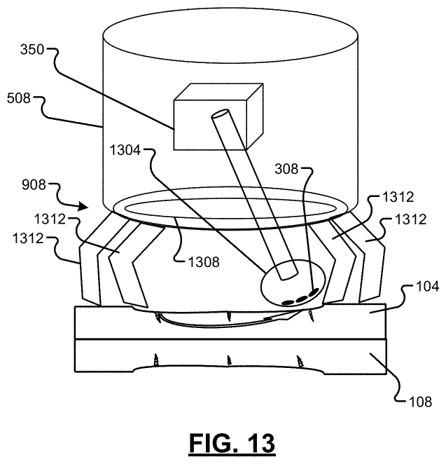

FIG. 13 includes a perspective side view including an example implementation of the probe 304 of FIG. 3 and the weld 112. In the example of FIG. 13, the inductor coils 308 are located on a pendulum 1304 that extends downward through an opening 1308 in the distal end 908 of the head portion 508. The actuator 350 swings the pendulum 1304 and therefore the inductor coils 308 (e.g., circularly, 360 degrees) around the weld 112. In this example, the inductor coils 308 are rotated around the weld 112, and the probe 304 does not need to be rotated.

In various implementations, the probe 304 may include legs that extend downwardly toward the weld 112 and away from the distal end 908 of the probe. For example, legs 1312 extend and contact the surface of the metal sheet outside of the weld 112. In this example, a circle defined by the legs 1312 has a diameter that is larger than the diameter of the weld 112. The diameter of the weld 112 may be the same from one spot weld to the next, such that the diameter of the circle defined by the legs 1312 will always be greater than any spot weld that is assessed via the probe 304.

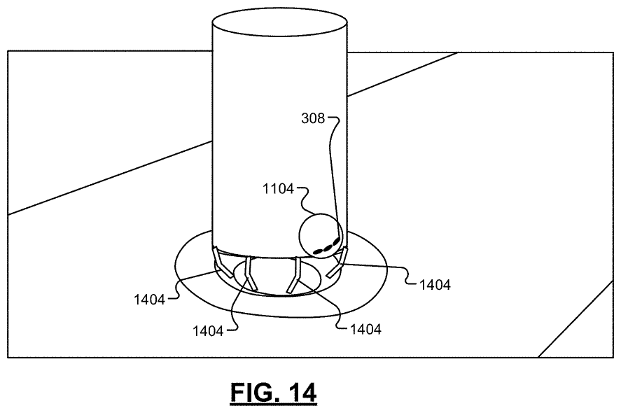

FIG. 14 includes a perspective side view including an example implementation of the probe 304 of FIG. 3 and the weld 112. As shown in the example of FIG. 14, the probe 304 may include legs 1404 that extend and contact the surface of the metal sheet inside of the weld 112. In this example, a circle defined by the legs 1404 has a diameter that is less than the diameter of the weld 112.

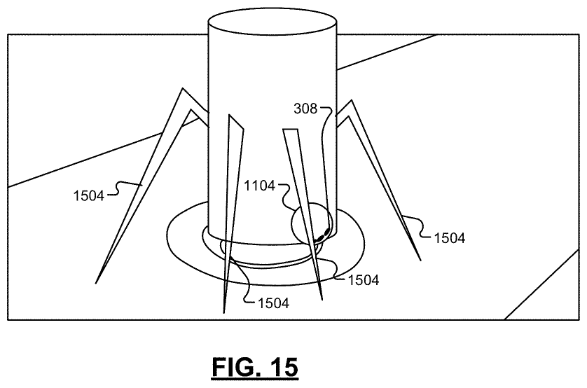

FIG. 15 includes a perspective side view including an example implementation of the probe 304 of FIG. 3 and the weld 112. As shown in the example of FIG. 15, the probe 304 may include legs 1504 that extend outwardly from the body portion 504 of the probe 304 and that contact the surface of the metal sheet outside of the weld 112. In this example, a circle defined by the legs 1504 has a diameter that is greater than the diameter of the weld 112.

As described above, the inductor coils 308 may include one or more than one inductor coil. FIGS. 16 and 17 include example arrays of multiple coils. Centers of the inductor coils 308 may be aligned vertically and horizontally (i.e., in two directions separated by 90 degrees), such as shown in the example of FIG. 16.

Centers of the inductor coils 308 may alternatively be staggered, such as shown in the example of FIG. 17. In the example of FIG. 17, the centers of the inductor coils 308 of a first row 1704 are not aligned vertically with the centers of the coils of a second row 1708. The centers of the inductor coils 308 of a third row 1712 are not vertically aligned with the centers of the coils of the second row 1708. The centers of the coils of each row, however, are horizontally aligned. More specifically, the centers of the inductor coils 308 of the first row 1704 are not aligned with the centers of the coils of the second row 1708 in any two directions that separated by 90 degrees. The centers of the inductor coils 308 of the third row 1712 are not aligned with the centers of the coils of the second row 1708 in any two directions that separated by 90 degrees.

The centers of the inductor coils 308 of the third row 1712 may or may not be vertically aligned with the centers of the coils of the first row 1704. While the example of the inductor coils 308 being arranged in three rows is provided, the inductor coils 308 may be arranged in one, two, three, or more than three rows. Arranging the inductor coils 308 in a staggered manner may increase a likelihood that a crack could not pass between adjacent ones of the inductor coils 308 without being detected.

The inductor coils 308 may be disposed upon a conformal polymer protective wear plate to help ensure that a consistent distance is maintained with varying curvatures in spot weld indentation. For example, the inductor coils 308 may be covered with a ceramic coating. The conformal polymer may be replaceable, such as over time as the conformal polymer experiences wear.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A probe for detecting discontinuities, the probe comprising:
a body portion;
a head portion;
one or more inductor coils located in the head portion and configured to:
receive power from a power source;
when power is received, induce an eddy current in a joint where a first metal component is joined with a second metal component;
based on the induced eddy current, output a signal indicative of an inductance of the one or more inductor coils; and
a plurality of positioning devices configured to maintain the one or more inductor coils approximately a predetermined distance from the surface.

2. The probe of claim 1 wherein the positioning devices include a plurality of extensions that extend only radially outwardly from the head portion.

3. The probe of claim 2 wherein the one or more inductor coils are located in one of the extensions.

4. The probe of claim 2 wherein the plurality of extensions include at least three extensions that extend only radially outwardly from the head portion,
wherein the at least three separate extensions are located equidistantly around the head portion.

5. The probe of claim 2 wherein the plurality of extensions define a circle having a first diameter that is greater than a second diameter of the joint.

6. The probe of claim 1 wherein the joint includes a spot weld formed by resistance spot welding (RSW).

7. The probe of claim 1 further comprising a central portion that is rotatable within the body portion,
wherein the head portion is coupled to and rotates with the central portion.

8. The probe of claim 1 wherein the plurality of positioning devices include rollers.

9. The probe of claim 8 wherein the rollers include one of ball casters, ball transfers, and Hudson bearings.

10. The probe of claim 8 wherein the rollers are spring loaded such that the rollers are biased away from the head portion.

11. The probe of claim 8 wherein the head portion defines a circle having a diameter, and
wherein all of the rollers are located within the diameter of the circle, and
wherein the diameter of the circle is one of less than and greater than a second diameter of the joint.

12. The probe of claim 8 wherein the head portion defines a circle having a diameter, and
wherein a plurality of the rollers are located outside of the diameter of the circle.

13. The probe of claim 1 further comprising a rotating portion configured to rotate radially inwardly and radially outwardly,
wherein the one or more inductor coils are located in the rotating portion.

14. The probe of claim 13 further comprising an actuator configured to rotate the rotating portion back and forth from a first predetermined radially inward position and a second predetermined radially outward position.

15. The probe of claim 14 wherein the actuator includes one of:
a motor; and
a rotating member and a multiple bar linkage that is coupled to the rotating member.

16. The probe of claim 1 further comprising a protective wear plate.

17. The probe of claim 1 wherein the positioning devices include a plurality of legs that extend radially outwardly from the probe and that are configured to extend toward the joint.

18. The probe of claim 17 wherein the legs define a circle having a first diameter that is one of less than and greater than a second diameter of the joint.

19. A method of detecting discontinuities, comprising:
locating a probe within a joint on a first metal component where the first metal component is joined with a second metal component,
wherein the probe includes:
one or more inductor coils configured to:
receive power from a power source;
when power is received, induce an eddy current in the joint; and
based on the induced eddy current, output a signal indicative of an inductance of the one or more inductor coils; and
a plurality of positioning devices configured to maintain the one or more inductor coils approximately a predetermined distance from the joint; and
rotating the probe within the joint while maintaining the positioning devices in contact with at least one of:
the joint; and
the first metal component.

20. The method of claim 19 further comprising:
generating a two-dimensional map of a region of interest within the joint based on the signal during the rotation of the probe within the joint.
